SEQUENCE LISTING (28 sequences)

SEQ ID NO: 1 - DNA, 1005 bp, Neisseria meningitidis
  source 1..1005: mol_type = genomic DNA
atgggccagccgctggttagcgttctgatctgcgcgtacaacgttgaaaaatatttcgcgcagagcctggcagctgttgttaaccagacctggcgtaacctggacattctgatcgttgatgatggctctaccgatggcaccctggcgatcgcgcagcgtttccaggaacaggacggtcgtatccgtattctggcgcagccgcgtaactctggtctgattccaagcctgaacatcggcctggatgaactggcgaaaagcggcggtggtggtgaatacatcgcgcgtaccgatgcggatgatatcgcagctccggattggattgaaaaaatcgttggtgaaatggaaaaagatcgtagcatcatcgcaatgggcgcttggctggaagtgctgtccgaagaaaaagatggcaaccgtctggcacgtcaccacgaacacggtaaaatctggaaaaaaccgacccgtcacgaagacatcgcggatttcttcccattcggcaacccgattcacaacaacaccatgatcatgcgtcgttccgtgatcgatggcggcctgcgttacaacaccgaacgtgattgggcagaagactatcagttctggtatgatgtttctaaactgggtcgtctggcgtactacccggaagcgctggttaaataccgtctgcacgctaaccaggttagctccaaatatagcatccgccagcacgaaatcgctcagggtatccagaaaaccgcacgtaacgatttcctgcagtctatgggtttcaaaacccgtttcgatagcctggaataccgtcagattaaagcggttgcgtatgaactgctggaaaaacacctgccggaagaagattttgaactggcgcgtcgtttcctgtaccagtgcttcaaacgtaccgataccctgccggcgggcgcttggctggatttcgcggcggatggccgtatgcgtcgtctgttcaccctgcgtcagtacttcggtatcctgcaccgtctgctgaaaaaccgttaa

SEQ ID NO: 2 - DNA, 846 bp, Neisseria meningitidis
  source 1..846: mol_type = genomic DNA
atgcaaaatcacgtcattagtcttgcgtcggccgcagaacgcagggcgcacattgccgataccttcggcaggcacggcatcccgtttcagtttttcgacgcactgatgccgtctgaaaggctggaacaggcaatggcggaactcgtccccggcttgtcggcgcacccctatttgagcggagtggaaaaagcctgctttatgagccacgccgtattgtggaagcaggcattggacgaaggtctgccgtatatcaccgtatttgaggacgacgttttactcggcgaaggtgcggaaaaattccttgccgaagacgcttggctgcaagaacgctttgacccggataccgcctttatcgtccgcttggaaacgatgtttatgcacgtcctgacctcgccctccggcgtggcggattactgcgggcgcgcctttccgctgttggaaagcgaacactgggggacggcgggctatatcatttcccgaaaagcgatgcggtttttcctggacaggtttgccgccctgccgcccgaagggctgcaccccgtcgatctgatgatgttcagcgattttttcgacagggaaggaatgccggtttgccagctcaatcccgccttgtgcgcccaagagctgcattatgccaagtttcacgaccaaaacagcgcattgggcagcctgatcgaacacgaccgcctcctgaaccgcaaacagcaaaggcgcgattcccccgccaacacattcaaacaccgcctgatccgcgccttgaccaaaatcagcagggaaagggaaaaacgccggcaaaggcgcgaacagttcattgtgccattccagcatcatcatcaccaccattaa

SEQ ID NO: 3 - DNA, 45 bp, synthetic construct
  source 1..45: mol_type = other DNA
tcgttttacaacgtcgtgacgttttagagctagaaatagcaagtt

SEQ ID NO: 4 - DNA, 45 bp, synthetic construct
  source 1..45: mol_type = other DNA
gtcacgacgttgtaaaacgaactagtattatacctaggactgagc

SEQ ID NO: 5 - DNA, 27 bp, synthetic construct
  source 1..27: mol_type = other DNA
tgaatgagggcatcgttcccactgcga

SEQ ID NO: 6 - DNA, 53 bp, synthetic construct
  source 1..53: mol_type = other DNA
ctcgagagctgtttcctgtgtgaaattgttatccgctcacaatttcacacaac

SEQ ID NO: 7 - DNA, 21 bp, synthetic construct
  source 1..21: mol_type = other DNA
ccattacggtcaatccgccgt

SEQ ID NO: 8 - DNA, 35 bp, synthetic construct
  source 1..35: mol_type = other DNA
acgctcatcgataatttcaccgccgaaaggcgcgg

SEQ ID NO: 9 - DNA, 52 bp, synthetic construct
  source 1..52: mol_type = other DNA
tcatcaccacagccaggatccaatgtactatttaaaaaacacaaacttttgg

SEQ ID NO: 10 - DNA, 45 bp, synthetic construct
  source 1..45: mol_type = other DNA
tgcggccgcaagcttgtcgacttaagcgacttcattcacctgacg

SEQ ID NO: 11 - DNA, 46 bp, synthetic construct
  source 1..46: mol_type = other DNA
tcatcaccacagccaggatccaatggcaatccacaatcgtgcaggc

SEQ ID NO: 12 - DNA, 44 bp, synthetic construct
  source 1..44: mol_type = other DNA
tgcggccgcaagcttgtcgacttacgcgtttttcagaacttcgc

SEQ ID NO: 13 - DNA, 45 bp, synthetic construct
  source 1..45: mol_type = other DNA
tcatcaccacagccaggatccaatgagagttctggttaccggtgg

SEQ ID NO: 14 - DNA, 42 bp, synthetic construct
  source 1..42: mol_type = other DNA
tgcggccgcaagcttgtcgacttaatcgggatatccctgtgg

SEQ ID NO: 15 - DNA, 45 bp, synthetic construct; identical to SEQ ID NO: 13
  source 1..45: mol_type = other DNA

SEQ ID NO: 16 - DNA, 46 bp, synthetic construct
  source 1..46: mol_type = other DNA
tgcggccgcaagcttgtcgacttacactccggattcgcgaaaatgg

SEQ ID NO: 17 - DNA, 45 bp, synthetic construct; identical to SEQ ID NO: 13
  source 1..45: mol_type = other DNA

SEQ ID NO: 18 - DNA, 45 bp, synthetic construct
  source 1..45: mol_type = other DNA
tgcggccgcaagcttgtcgactcagcactgtcctgctccttgtga

SEQ ID NO: 19 - DNA, 39 bp, synthetic construct
  source 1..39: mol_type = other DNA
tcatcaccacagccaggatccaatgggccagccgctggt

SEQ ID NO: 20 - DNA, 43 bp, synthetic construct
  source 1..43: mol_type = other DNA
tgcggccgcaagcttgtcgacttaacggtttttcagcagacgg

SEQ ID NO: 21 - DNA, 47 bp, synthetic construct
  source 1..47: mol_type = other DNA
agatatacatatggcagatctaatgcaaaatcacgtcattagtcttg

SEQ ID NO: 22 - DNA, 45 bp, synthetic construct
  source 1..45: mol_type = other DNA
ggtttctttaccagactcgagttaatggtggtgatgatgatgctg

SEQ ID NO: 23 - DNA, 3075 bp, Escherichia coli
  source 1..3075: mol_type = genomic DNA
atgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataa

SEQ ID NO: 24 - DNA, 1641 bp, Escherichia coli
  source 1..1641: mol_type = genomic DNA
atggcaatccacaatcgtgcaggccaacctgcacaacagagtgatttgattaacgtcgcccaactgacggcgcaatattatgtactgaaaccagaagcagggaatgcggagcacgcggtgaaattcggtacttccggtcaccgtggcagtgcagcgcgccacagctttaacgagccgcacattctggcgatcgctcaggcaattgctgaagaacgtgcgaaaaacggcatcactggcccttgctatgtgggtaaagatactcacgccctgtccgaacctgcattcatttccgttctggaagtgctggcagcgaacggcgttgatgtcattgtgcaggaaaacaatggcttcaccccgacgcctgccgtttccaatgccatcctggttcacaataaaaaaggtggcccgctggcagacggtatcgtgattacaccgtcccataacccgccggaagatggtggaatcaaatacaatccgccaaatggtggcccggctgataccaacgtcactaaagtggtggaagacagggccaacgcactgctggccgatggcctgaaaggcgtgaagcgtatctccctcgacgaagcgatggcatccggtcatgtgaaagagcaggatctggtgcagccgttcgtggaaggtctggccgatatcgttgatatggccgcgattcagaaagcgggcctgacgctgggcgttgatccgctgggcggttccggtatcgaatactggaagcgtattggcgagtattacaacctcaacctgactatcgttaacgatcaggtcgatcaaaccttccgctttatgcaccttgataaagacggcgcgatccgtatggactgctcctccgagtgtgcgatggcgggcctgctggcactgcgtgataagttcgatctggcgtttgctaacgacccggattatgaccgtcacggtatcgtcactccggcaggtttgatgaatccgaaccactacctggcggtggcaatcaattacctgttccagcatcgtccgcagtggggcaaagatgttgccgtcggtaaaacgctggtttcatctgcgatgatcgaccgtgtggtcaacgacttgggccgtaaactggtagaagtcccggtaggtttcaaatggtttgtcgatggtctgttcgacggcagcttcggctttggcggcgaagagagtgcaggggcttccttcctgcgtttcgacggcacgccgtggtccaccgacaaagacggcatcatcatgtgtctgctggcggcggaaatcaccgctgtcaccggtaagaacccgcaggaacactacaacgaactggcaaaacgctttggtgcgccgagctacaaccgtttgcaggcagctgcgacttccgcacaaaaagcggcgctgtctaagctgtctccggaaatggtgagcgccagcaccctggcaggtgacccgatcaccgcgcgcctgactgctgctccgggcaacggtgcttctattggcggtctgaaagtgatgactgacaacggctggttcgccgcgcgtccgtcaggcacggaagacgcatataagatctactgcgaaagcttcctcggtgaagaacatcgcaagcagattgagaaagaagcggttgagattgttagcgaagttctgaaaaacgcgtaa

SEQ ID NO: 25 - DNA, 1017 bp, Escherichia coli
  source 1..1017: mol_type = genomic DNA
atgagagttctggttaccggtggtagcggttacattggaagtcatacctgtgtgcaattactgcaaaacggtcatgatgtcatcattcttgataacctctgtaacagtaagcgcagcgtactgcctgttatcgagcgtttaggcggcaaacatccaacgtttgttgaaggcgatattcgtaacgaagcgttgatgaccgagatcctgcacgatcacgctatcgacaccgtgatccacttcgccgggctgaaagccgtgggcgaatcggtacaaaaaccgctggaatattacgacaacaatgtcaacggcactctgcgcctgattagcgccatgcgcgccgctaacgtcaaaaactttatttttagctcctccgccaccgtttatggcgatcagcccaaaattccatacgttgaaagcttcccgaccggcacaccgcaaagcccttacggcaaaagcaagctgatggtggaacagatcctcaccgatctgcaaaaagcccagccggactggagcattgccctgctgcgctacttcaacccggttggcgcgcatccgtcgggcgatatgggcgaagatccgcaaggcattccgaataacctgatgccatacatcgcccaggttgctgtaggccgtcgcgactcgctggcgatttttggtaacgattatccgaccgaagatggtactggcgtacgcgattacatccacgtaatggatctggcggacggtcacgtcgtggcgatggaaaaactggcgaacaagccaggcgtacacatctacaacctcggcgctggcgtaggcaacagcgtgctggacgtggttaatgccttcagcaaagcctgcggcaaaccggttaattatcattttgcaccgcgtcgcgagggcgaccttccggcctactgggcggacgccagcaaagccgaccgtgaactgaactggcgcgtaacgcgcacactcgatgaaatggcgcaggacacctggcactggcagtcacgccatccacagggatatcccgattaa

SEQ ID NO: 26 - DNA, 1047 bp, Escherichia coli
  source 1..1047: mol_type = genomic DNA
atgacgcaatttaatcccgttgatcatccacatcgccgctacaacccgctcaccgggcaatggattctggtttcaccgcaccgcgctaagcgcccctggcagggggcgcaggaaacgccagccaaacaggtgttacctgcgcacgatccagattgcttcctctgcgcaggtaatgtgcgggtgacaggcgataaaaaccccgattacaccgggacttacgttttcactaatgactttgcggctttgatgtctgacacgccagatgcgccagaaagtcacgatccgctgatgcgttgccagagcgcgcgcggcaccagccgggtgatctgcttttcaccggatcacagtaaaacgctgccagagctcagcgttgcagcattgacggaaatcgtcaaaacctggcaggagcaaaccgcagaactggggaaaacgtacccatgggtgcaggtttttgaaaacaaaggcgcggcgatgggctgctctaacccgcatccgcacggtcagatttgggcaaatagcttcctgcctaacgaagctgagcgcgaagaccgcctgcaaaaagaatattttgccgaacagaaatcaccaatgctggtggattatgttcagcgcgagctggcagacggtagccgtaccgttgtcgaaaccgaacactggttagccgtcgtgccttactgggctgcctggccgttcgaaacgctactgctgcccaaagcccacgttttacggatcaccgatttgaccgacgcccagcgcagcgatctggcgctggcgttgaaaaagctgaccagtcgttatgacaacctcttccagtgctccttcccctactctatgggctggcacggcgcgccatttaatggcgaagagaatcaacactggcagctgcacgcgcacttttatccgcctctgctgcgctccgccaccgtacgtaaatttatggttggttatgaaatgctggcagagacccagcgagacctgaccgcagaacaggcagcagagcgtttgcgcgcagtcagcgatatccattttcgcgaatccggagtgtaa

SEQ ID NO: 27 - DNA, 1149 bp, Escherichia coli
  source 1..1149: mol_type = genomic DNA
atgagtctgaaagaaaaaacacaatctctgtttgccaacgcatttggctaccctgccactcacaccattcaggcgcctggccgcgtgaatttgattggtgaacacaccgactacaacgacggtttcgttctgccctgcgcgattgattatcaaaccgtgatcagttgtgcaccacgcgatgaccgtaaagttcgcgtgatggcagccgattatgaaaatcagctcgacgagttttccctcgatgcgcccattgtcgcacatgaaaactatcaatgggctaactacgttcgtggcgtggtgaaacatctgcaactgcgtaacaacagcttcggcggcgtggacatggtgatcagcggcaatgtgccgcagggtgccgggttaagttcttccgcttcactggaagtcgcggtcggaaccgtattgcagcagctttatcatctgccgctggacggcgcacaaatcgcgcttaacggtcaggaagcagaaaaccagtttgtaggctgtaactgcgggatcatggatcagctaatttccgcgctcggcaagaaagatcatgccttgctgatcgattgccgctcactggggaccaaagcagtttccatgcccaaaggtgtggctgtcgtcatcatcaacagtaacttcaaacgtaccctggttggcagcgaatacaacacccgtcgtgaacagtgcgaaaccggtgcgcgtttcttccagcagccagccctgcgtgatgtcaccattgaagagttcaacgctgttgcgcatgaactggacccgatcgtggcaaaacgcgtgcgtcatatactgactgaaaacgcccgcaccgttgaagctgccagcgcgctggagcaaggcgacctgaaacgtatgggcgagttgatggcggagtctcatgcctctatgcgcgatgatttcgaaatcaccgtgccgcaaattgacactctggtagaaatcgtcaaagctgtgattggcgacaaaggtggcgtacgcatgaccggcggcggatttggcggctgtatcgtcgcgctgatcccggaagagctggtgcctgccgtacagcaagctgtcgctgaacaatatgaagcaaaaacaggtattaaagagactttttacgtttgtaaaccatcacaaggagcaggacagtgctga

SEQ ID NO: 28 - DNA, 1254 bp, Escherichia coli
  source 1..1254: mol_type = genomic DNA
atgtactatttaaaaaacacaaacttttggatgttcggtttattctttttcttttacttttttatcatgggagcctacttcccgtttttcccgatttggctacatgacatcaaccatatcagcaaaagtgatacgggtattatttttgccgctatttctctgttctcgctattattccaaccgctgtttggtctgctttctgacaaactcgggctgcgcaaatacctgctgtggattattaccggcatgttagtgatgtttgcgccgttctttatttttatcttcgggccactgttacaatacaacattttagtaggatcgattgttggtggtatttatctaggcttttgttttaacgccggtgcgccagcagtagaggcatttattgagaaagtcagccgtcgcagtaatttcgaatttggtcgcgcgcggatgtttggctgtgttggctgggcgctgtgtgcctcgattgtcggcatcatgttcaccatcaataatcagtttgttttctggctgggctctggctgtgcactcatcctcgccgttttactctttttcgccaaaacggatgcgccctcttctgccacggttgccaatgcggtaggtgccaaccattcggcatttagccttaagctggcactggaactgttcagacagccaaaactgtggtttttgtcactgtatgttattggcgtttcctgcacctacgatgtttttgaccaacagtttgctaatttctttacttcgttctttgctaccggtgaacagggtacgcgggtatttggctacgtaacgacaatgggcgaattacttaacgcctcgattatgttctttgcgccactgatcattaatcgcatcggtgggaaaaacgccctgctgctggctggcactattatgtctgtacgtattattggctcatcgttcgccacctcagcgctggaagtggttattctgaaaacgctgcatatgtttgaagtaccgttcctgctggtgggctgctttaaatatattaccagccagtttgaagtgcgtttttcagcgacgatttatctggtctgtttctgcttctttaagcaactggcgatgatttttatgtctgtactggcgggcaatatgtatgaaagcatcggtttccagggcgcttatctggtgctgggtctggtggcgctgggcttcaccttaatttccgtgttcacgcttagcggccccggcccgctttccctgctgcgtcgtcaggtgaatgaagtcgcttaa

(19) United States
(12) Patent Application Publication
(10) Pub. No.: US 2023/0279456 A1
(43) Pub. Date: Sep. 7, 2023

(54) Genetically Engineered Bacteria Producing Lacto-N-neotetraose and Production Method Thereof

(71) Applicant: Jiangnan University, Wuxi (CN)

(72) Inventors: Tao ZHANG, Wuxi (CN); Bo JIANG, Wuxi (CN); Miaomiao HU, Wuxi (CN); Mengli LI, Wuxi (CN)

(21) Appl. No.: 18/061,585

(22) Filed: Dec. 5, 2022

Related U.S. Application Data
(63) Continuation of application No. PCT/CN2022/087318, filed on Apr. 18, 2022.

(30) Foreign Application Priority Data
Apr. 25, 2021 (CN) 2021104470824

(51) Int. Cl. C12P 19/26; C12N 15/70; C12N 9/10; C12N 9/12; C12N 9/38; C12N 9/90

(52) U.S. Cl. CPC C12P 19/26; C12N 15/70; C12N 9/1051; C12N 9/1205; C12N 9/1241; C12N 9/2471; C12N 9/90; C12Y 204/01038; C12Y 204/01222; C12Y 207/01006; C12Y 501/03002; C12Y 504/02002; C12Y 207/07012

(57) ABSTRACT

The disclosure discloses genetically engineered bacteria producing lacto-N-neotetraose and a production method thereof, and belongs to the fields of metabolic engineering and food biotechnology. To solve the problem of low yield of lacto-N-neotetraose produced by a microbial method in the prior art, through exogenous expression of lgtA and lgtB, reasonable combination and regulation of overexpression of lacY, pgm, galE, galT and galK in a lacto-N-neotetraose synthesis pathway, knockout of lacZ expression in an Escherichia coli host, and optimization of a carbon source in the culture process, the disclosure achieves the objectives of regulating the carbon flux of a metabolic pathway and improving the yield of lacto-N-neotetraose. In a shake flask experiment, the yield of lacto-N-neotetraose produced by E. coli increased from 304 mg/L to 1031 mg/L, laying a foundation for industrial production of the lacto-N-neotetraose.

### REFERENCE TO SEQUENCE LISTING

The instant application contains a Sequence Listing in XML format as a file named “3050-YGHY-2022-64.xml”, created on Nov. 11, 2022, last modified on May 24, 2023, of 36.2 kB in size, and which is hereby incorporated by reference in its entirety.

### TECHNICAL FIELD

The disclosure relates to genetically engineered bacteria producing lacto-N-neotetraose and a production method thereof, and belongs to the technical fields of metabolic engineering and food fermentation.

### BACKGROUND

The content of human milk oligosaccharides (HMOs) is the third largest in solid components of human milk after lactose and fat, and is 12-13 g/L in mature milk and 22-23 g/L in colostrum, to which cow milk is incomparable (the content of cow milk oligosaccharides is less than 1 g/L). HMOs can tolerate hydrolysis of enzymes in the digestive tract of infants, and then resist infection of pathogenic microorganisms in the gastrointestinal tract and maintain microecological balance of the gastrointestinal tract. Lacto-N-neotetraose, as an important oligosaccharide in HMOs, has biological functions such as enhancing human immunity, regulating intestinal flora, promoting cell maturation and accelerating wound healing. In view of the important biological functions and physiological activity, lacto-N-neotetraose has been allowed to be added into commercial infant formula. However, the amount of lacto-N-neotetraose separated and extracted from natural products is very small, far less than the need for research. Therefore, it is the best choice to obtain lacto-N-neotetraose by artificial synthesis.

At present, the reported synthesis methods of lacto-N-neotetraose mainly include chemical synthesis, enzymatic synthesis and fermentation synthesis. Chemical synthesis has the problems such as complex reaction steps and expensive raw materials, resulting in high production costs and being not conducive to large-scale industrial synthesis. In addition, some toxic reagents used in chemical synthesis make the product unsuitable for food additives. Enzymatic synthesis uses expensive nucleotide sugars as substrates and is not conducive to large-scale production of lacto-N-neotetraose. Biological synthesis may use cheap carbon and nitrogen sources and substrates to achieve the synthesis of lacto-N-neotetraose without affecting the environment. Therefore, preparation of lacto-N-neotetraose by biological synthesis has attracted more and more attention.

### SUMMARY

The disclosure provides genetically engineered bacteria producing lacto-N-neotetraose, wherein in the genetically engineered bacteria, β-galactosidase gene lacZ is knocked out, and β-1,3-acetylglucosamine transferase gene lgtA, β-1,4-galactosyltransferase gene lgtB, phosphoglucomutase gene pgm, UDP-glucose-4-epimerase gene galE, galactose-1-phosphate uridyltransferase gene galT, galactokinase gene galK and β-galactoside permease gene lacY are overexpressed.

In one embodiment, the β-1,3-acetylglucosamine transferase gene lgtA and β-1,4-galactosyltransferase gene lgtB are both from Neisseria meningitidis; the nucleotide sequence of β-1,3-acetylglucosamine transferase gene lgtA is shown in SEQ ID NO: 1; and the nucleotide sequence of β-1,4-galactosyltransferase gene lgtB is shown in SEQ ID NO: 2.

In one embodiment, the phosphoglucomutase gene pgm, UDP-glucose-4-epimerase gene galE, galactose-1-phosphate uridyltransferase gene galT, galactokinase gene galK, β-galactoside permease gene lacY and β-galactosidase gene lacZ are all from Escherichia coli K-12; the Gene ID of the phosphoglucomutase gene pgm is 945271 (the nucleotide sequence of which is shown in SEQ ID NO: 24); the Gene ID of the UDP-glucose-4-epimerase gene galE is 945354 (the nucleotide sequence of which is shown in SEQ ID NO: 25); the Gene ID of the galactose-1-phosphate uridyltransferase gene galT is 945357 (the nucleotide sequence of which is shown in SEQ ID NO: 26); the Gene ID of the galactokinase gene galK is 945358 (the nucleotide sequence of which is shown in SEQ ID NO: 27); the Gene ID of the β-galactoside permease gene lacY is 949083 (the nucleotide sequence of which is shown in SEQ ID NO: 28); and the Gene ID of the β-galactosidase gene lacZ is 945006 (the nucleotide sequence of which is shown in SEQ ID NO: 23).

In one embodiment, the genetically engineered bacteria express the gene lacY with pETDuet-1 plasmid, express the genes pgm, galE, galT and galK sequentially with pRSFDuet-1 plasmid, and express the genes lgtA and lgtB sequentially with pCDFDuet-1 plasmid.

The disclosure further provides a method for producing lacto-N-neotetraose, including the following steps:
(1) culturing the genetically engineered bacteria in a seed medium to obtain a seed solution;
(2) inoculating the seed solution into a fermentation system and culturing the seed solution until the OD₆₀₀ is 0.6-0.8; and
(3) adding IPTG with a final concentration of 0.4 mM and lactose with a final concentration of 8 g/L-10 g/L for performing induction to obtain a fermentation broth containing lacto-N-neotetraose.

In one embodiment, carbon source in the fermentation medium is one or more of glucose, galactose and glycerin.

Preferably, the carbon source is glycerin or a mixture of glycerin and galactose.

More preferably, the carbon source is 20 g/L glycerin, or a mixture of 10 g/L glycerin and 10 g/L galactose.

In one embodiment, the fermentation system further contains 13.5 g/L potassium dihydrogen phosphate, 4.0 g/L diammonium hydrogen phosphate, 1.7 g/L citric acid, 1.4 g/L magnesium sulfate heptahydrate and 10 ml/L trace metal elements, the trace metal elements including 10 g/L ferrous sulfate, 2.25 g/L zinc sulfate heptahydrate, 1.0 g/L anhydrous copper sulfate and 2.0 g/L calcium chloride dihydrate, and the pH of the fermentation system being 6.8.

In one embodiment, the seed medium in the method is an LB liquid medium, and the seeds are cultured at 35° C.-40° C. and 200 rpm-250 rpm for 10 h-14 h in a shake flask.

In one embodiment, the fermentation broth in the method is specifically prepared by inoculating the seed solution into a fermentation system, culturing the seed solution at 35° C.-40° C. and 200 rpm-250 rpm until the OD₆₀₀ is 0.6-0.8, then adding IPTG with a final concentration of 0.4 mM and lactose with a final concentration of 8 g/L-10 g/L, and inducing culture at 22° C.-30° C. and 200 rpm-250 rpm for 42 h-48 h.

The disclosure further provides application of the genetically engineered bacteria in producing lacto-N-neotetraose.

Through exogenous expression of lgtA and lgtB, reasonable combination and regulation of overexpression of lacY, pgm, galE, galT and galK in a lacto-N-neotetraose synthesis pathway, knockout of lacZ expression in the lacto-N-neotetraose synthesis pathway of an E. coli host, and optimization of a carbon source of a fermentation medium, the disclosure achieves the objectives of regulating the carbon flux of a metabolic pathway and improving the yield of lacto-N-neotetraose. In a shake flask experiment, the yield of lacto-N-neotetraose produced by E. coli increased from 304 mg/L to 1031 mg/L, laying a foundation for industrial production of the lacto-N-neotetraose.

### BRIEF DESCRIPTION OF FIGURES

FIG. 1 shows a diagram of the metabolic pathway of lacto-N-neotetraose.

FIG. 2A shows a secondary mass spectrogram of a lacto-N-neotetraose standard sample.

FIG. 2B shows a secondary mass spectrogram of a lacto-N-neotetraose product sample.

### DETAILED DESCRIPTION

The specific implementation of the disclosure is further described in the following examples and figures. Plasmids, PCR reagents, restriction endonucleases, plasmid extraction kits, DNA gel extraction kits, and the like used in the following examples are commercial products, and are specifically operated according to the kit instructions. The embodiments of the disclosure are not limited thereto, and other unspecified experimental operations and process parameters are carried out according to conventional technology.

Plasmids and DNA products are sequenced by Talen Biotechnology (Shanghai) Co., Ltd.

E. coli competent cells are prepared using a kit of Shanghai Sangon Biotechnology Co., Ltd.

An LB liquid medium contains 10 g/L peptone, 5 g/L yeast extract, and 10 g/L sodium chloride.

An LB solid medium contains 10 g/L peptone, 5 g/L yeast extract powder, 10 g/L sodium chloride, and 15 g/L agar powder.

The lacto-N-neotetraose in the embodiments of the disclosure is measured by HPLC, specifically:

1 mL of fermentation broth is boiled at 100° C. for 10 min and centrifuged at 13400 rpm for 10 min, the supernatant is filtered with a 0.22 μM membrane, and the yield of lacto-N-neotetraose is detected by HPLC. HPLC detection uses a differential refraction detector, the chromatographic column is Rezex ROA organic acid (Phenomenex, USA), the column temperature is 50° C., the mobile phase is a 5 mM H₂SO₄ aqueous solution, the flow rate is 0.6 mL/min, and the injection volume is 10 μL.

### Example 1 Knockout of Gene lacZ from Genome of E. coli BL21 (DE3)

LacZ was knocked out from E. coli BL21 (lacZ has a gene ID of 945006 and is shown in SEQ ID NO: 23) using a CRISPR-Cas9 gene knockout system by the following specific steps (the primer sequences involved are shown in Table 1):
(1) The upstream and downstream fragments of lacZ were amplified by PCR using lacZ-up-FIR and lacZ-down-F/R respectively and the genome of E. coli BL21 as a template, and a DNA fragment was extracted from gel. Then a complete lacZ template was obtained by overlapping PCR using a lacZ-up-F/lacZ-down-R primer and the lacZ upstream and downstream fragments as templates respectively, and a DNA fragment was extracted from gel.
(2) An N20 sequence on an original plasmid was replaced with an N20 sequence complementary to the lacZ sequence by PCR amplification using the original pTargetF plasmid as a template and lacZ-sg-F/R as a primer, and a pTargetF plasmid targeting lacZ was obtained (the constructed plasmid was named pTargetF-lacZ, and the targeting plasmid information was a targeting plasmid pTargetF with the lacZ specific N20 sequence). The targeting plasmid was transformed into E. coli DH5a competent cells, spread on an LB plate (containing spectinomycin), cultured in a large scale at 37° C., extracted and sequenced.
(3) PCas plasmids and E. coli BL21 competent cells were placed on ice for 5 min until the competent cells melted. 5 μL of plasmids were added to 100 μL of competent cells, gently mixed, placed in an ice bath for 30 min, thermally activated at 42° C. for 90 s, and immediately placed on ice for 5 min. The mixture was added to 1 mL of LB medium and cultured at 30° C. and 180 rpm for 1 h. 200 μL of concentrated bacterial solution was taken and evenly spread on an LB plate (containing kanamycin), and cultured upside down at 30° C. overnight until a single colony of E. coli BL21/pCas grows.
(4) The single colony of E. coli BL21/pCas was picked and cultured in an LB medium at 30° C. for 1.0 h, and L-arabinose with a final concentration of 30 mM was added to induce expression of a pCas-λ-red system. When the OD₆₀₀ reached 0.6-0.8, E. coli BL21/pCas competent cells were prepared.
(5) 400 ng of pTargetF plasmids and 1000 ng of donor DNA fragments (i.e., lacZ template fragments obtained in step 1) were electro-transformed into the E. coli BL21/pCas competent cells obtained in step (4), spread on an LB plate (containing kanamycin and spectinomycin), and cultured at 30° C. for 24 h. Positive colonies on the plate were picked, cultured in an LB medium for 10 h, and sent to Talen Biotechnology (Shanghai) Co., Ltd. for sequencing verification.
(6) The positive cloned colonies obtained in step (5) were picked into a 4 ml LB liquid test tube, IPTG with a final concentration of 1 mM and 30 mg/L kanamycin were added, and the cells were cultured at 30° C. for 8-16 h to remove the pTargetF plasmids, and then cultured at 42° C. for 12 h to remove the pCas plasmids.

TABLE 1
Primer sequences for lacZ knockout

| Primer name | Sequence NO. | Primer sequences (5′-3′) |
|---|---|---|
| lacZ-sg-F | SEQ ID NO: 3 | TCGTTTTACAACGTCGTGACGTTTTAGAGCTAGAAATAGCAAGTT |
| lacZ-sg-R | SEQ ID NO: 4 | GTCACGACGTTGTAAAACGAACTAGTATTATACCTAGGACTGAGC |
| lacZ-up-F | SEQ ID NO: 5 | TGAATGAGGGCATCGTTCCCACTGCGA |
| lacZ-up-R | SEQ ID NO: 6 | CTCGAGAGCTGTTTCCTGTGTGAAATTGTTATCCGCTCACAATTTCA |
| | | CACAAC |
| lacZ-down-F | SEQ ID NO: 7 | CCATTACGGTCAATCCGCCGT |
| lacZ-down-R | SEQ ID NO: 8 | ACGCTCATCGATAATTTCACCGCCGAAAGGCGCGG |

### Example 2 Construction of Recombinant Bacteria and Screening of Plasmid Combinations

Recombinant bacteria are constructed specifically by the following steps (the primer sequences involved are shown in Table 2):
(1) Acquisition of lacY gene fragment (the Gene ID of lacY is 949083): A lacY gene fragment was amplified by PCR using the genome of E. coli K-12 as a template and lacY-F/lacY-R as a primer, and a DNA fragment was extracted from gel. The extracted DNA gene fragment was ligated to the BamHI and SalI restriction sites of the vector pETDuet-1 through a seamless cloning kit (Nanjing Vazyme Biotech Co., Ltd), and finally the plasmid pET-lacY was acquired.
(2) Acquisition of pgm, galE, galE-galT and galE-galT-galK gene fragments (the Gene ID of pgm is 945271, the Gene ID of galE is 945354, the Gene ID of galT is 945357 and the Gene ID of galK is 945358): A pgm gene fragment was amplified by PCR using the genome of E. coli K-12 as a template and pgm-F/pgm-R as a primer, and a DNA fragment was extracted from gel; a galE gene cluster fragment was amplified by PCR using galE-F/galE-R as a primer, and a DNA fragment was extracted from gel; a galE-galT gene cluster fragment was amplified by PCR using galET-F/galET-R as a primer, and a DNA fragment was extracted from gel; and a galE-galT-galK gene cluster fragment was amplified by PCR using galETK-F/galETK-R as a primer, and a DNA fragment was extracted from gel. The extracted DNA gene fragments were ligated to the BamHI, SalI, BgiII and XhoI restriction sites of the vector pRSFDuet-1 respectively through a seamless cloning kit (Nanjing Vazyme Biotech Co., Ltd), and finally the plasmids pRSF-pgm, pRSF-pgm-galE, pRSF-pgm-galET and pRSF-pgm-galETK were acquired.
(3) Acquisition of lgtA and lgtB gene fragments: The gene sequences of lgtA and lgtB (the nucleotide sequence of lgtA is shown in SEQ ID NO: 1, and the nucleotide sequence of lgtB is shown in SEQ ID NO: 2) of N. meningitidis were found out and synthesized by entrusting Talen Biotechnology (Shanghai) Co., Ltd. By means of the BamHI, SalI, BgiII and XhoI restriction sites, the synthesized gene fragment was ligated to the BamHI, SalI, BgiII and XhoI restriction sites of the vector pCDFDuet-1 through a seamless cloning kit (Nanjing Vazyme Biotech Co., Ltd), and finally the plasmid pCDF-lgtA-lgtB was acquired.

TABLE 2
Primers for plasmid construction

| Primer | | |
|---|---|---|
| name | Sequence NO. | Primer sequences (5′-3′) |
| lacY-F | SEQ ID NO: 9 | TCATCACCACAGCCAGGATCCAATGTACTATTTAAAAAACACAAACTTTTGG |
| lacY-R | SEQ ID NO: 10 | TGCGGCCGCAAGCTTGTCGACTTAAGCGACTTCATTCACCTGACG |
| pgm-F | SEQ ID NO: 11 | TCATCACCACAGCCAGGATCCAATGGCAATCCACAATCGTGCAGGC |
| pgm-R | SEQ ID NO: 12 | TGCGGCCGCAAGCTTGTCGACTTACGCGTTTTTCAGAACTTCGC |
| galE-F | SEQ ID NO: 13 | TCATCACCACAGCCAGGATCCAATGAGAGTTCTGGTTACCGGTGG |
| galE-R | SEQ ID NO: 14 | TGCGGCCGCAAGCTTGTCGACTTAATCGGGATATCCCTGTGG |
| galET-F | SEQ ID NO: 15 | TCATCACCACAGCCAGGATCCAATGAGAGTTCTGGTTACCGGTGG |
| galET-R | SEQ ID NO: 16 | TGCGGCCGCAAGCTTGTCGACTTACACTCCGGATTCGCGAAAATGG |
| galETK-F | SEQ ID NO: 17 | TCATCACCACAGCCAGGATCCAATGAGAGTTCTGGTTACCGGTGG |
| galETK-R | SEQ ID NO: 18 | TGCGGCCGCAAGCTTGTCGACTCAGCACTGTCCTGCTCCTTGTGA |
| lgtA-F | SEQ ID NO: 19 | TCATCACCACAGCCAGGATCCAATGGGCCAGCCGCTGGT |
| lgtA-R | SEQ ID NO: 20 | TGCGGCCGCAAGCTTGTCGACTTAACGGTTTTTCAGCAGACGG |
| lgtB-F | SEQ ID NO: 21 | AGATATACATATGGCAGATCTAATGCAAAATCACGTCATTAGTCTTG |
| lgtB-R | SEQ ID NO: 22 | GGTTTCTTTACCAGACTCGAGTTAATGGTGGTGATGATGATGCTG |

(4) The plasmids pET-lacY, pRSF-pgm, pRSF-pgm-galE, pRSF-pgm-galET, pRSF-pgm-galETK and pCDF-lgtA-lgtB acquired by the above steps were combined with different number of key genes in the synthetic pathway of lacto-N-neotetraose, and 6 different engineered strains represented as A0, A1, A2, A3, A4 and A5 respectively were obtained (see Table 3).

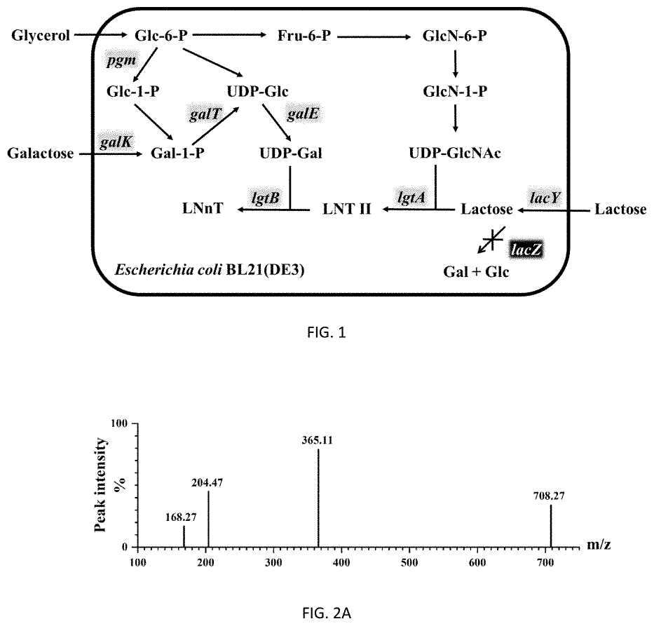

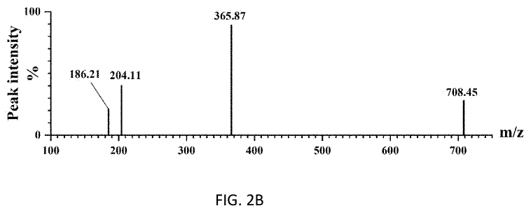

After fermentation, the yields of lacto-N-neotetraose of different engineered strains were 304 mg/L, 508 mg/L, 595 mg/L, 654 mg/L, 749 mg/L and 837 mg/L respectively. Compared with A0, the yield of lacto-N-neotetraose of strain A5 increased by 175.3%. Therefore, the expression of the endogenous genes pgm and galE-galT-galK of E. coli related to UDP-galactose synthesis can increase the yield of lacto-N-neotetraose. The metabolic pathway of lacto-N-neotetraose of the strain A5 is shown in FIG. 1.

The fermentation method was as follows: the constructed genetically engineered strains A0-A5 were inoculated into LB liquid media, and the strains were cultured at 37° C. and 200 rpm for 12 h in a shake flask to obtain seed solutions; the seed solutions were inoculated into 50 ml of fermentation media at an inoculation volume of 2 mL/100 mL, and the seed solutions were cultured at 37° C. and 200 rpm in a shake flask until the OD₆₀₀ was 0.6; and IPTG with a final concentration of 0.4 mM was added, lactose was added until the lactose concentration was 10 g/L, and culture was induced at 25° C. and 200 rpm for 48 h to obtain the fermentation broths.

The above fermentation medium contains 20 g/L glycerin, 13.5 g/L potassium dihydrogen phosphate, 4.0 g/L diammonium hydrogen phosphate, 1.7 g/L citric acid, 1.4 g/L magnesium sulfate heptahydrate and 10 ml/L trace metal elements, the trace metal elements including 10 g/L ferrous sulfate, 2.25 g/L zinc sulfate heptahydrate, 1.0 g/L anhydrous copper sulfate and 2.0 g/L calcium chloride dihydrate, and the pH of the fermentation medium being 6.8.

TABLE 3
Details of engineered strains

| | | Yield of |
|---|---|---|
| | | lacto-N- |
| Strain | | neotetraose |
| name | Plasmids and genotypes contained in the host | (mg/L) |
| A0 | E. coli BL21 (DE3) with lacZ gene knocked out, | 304 |
| | containing plasmid pCDF-IgtA-IgtB | |
| A1 | E. coli BL21 (DE3) with lacZ gene knocked out, | 508 |
| | containing plasmids pCDF-IgtA-IgtB and pET-lacY | |
| A2 | E. coli BL21 (DE3) with lacZ gene knocked out, | 595 |
| | containing plasmids pCDF-IgtA-IgtB, pET-lacY and | |
| | pRSF-pgm | |
| A3 | E. coli BL21 (DE3) with lacZ gene knocked out, | 654 |
| | containing plasmids pCDF-IgtA-IgtB, pET-lacY and | |
| | pRSF-pgm-galE | |
| A4 | E. coli BL21 (DE3) with lacZ gene knocked out, | 749 |
| | containing plasmids pCDF-IgtA-IgtB, pET-lacY and | |
| | pRSF-pgm-galE-galT | |
| A5 | E. coli BL21 (DE3) with lacZ gene knocked out, | 837 |
| | containing plasmids pCDF-IgtA-IgtB, pET-lacY and | |
| | pRSF-pgm-galE-galT-galK | |

### Example 3: Screening of Different Combinations of Carbon Sources for Producing Lacto-N-Neotetraose

The engineered strain A5 with high lacto-N-neotetraose yield obtained in Example 2 was fermented in fermentation media with different combinations of carbon sources by the following specific fermentation method:

The constructed genetically engineered strain A5 was inoculated into an LB liquid medium, and the strain was cultured at 37° C. and 200 rpm for 12 h in a shake flask to obtain a seed solution; the seed solution were inoculated into 50 ml of fermentation media at an inoculation volume of 2 mL/100 mL, and the seed solution was cultured at 37° C. and 200 rpm in a shake flask until the OD₆₀₀ was 0.6; and IPTG with a final concentration of 0.4 mM was added, lactose was added until the lactose concentration was 10 g/L, and culture was induced at 25° C. and 200 rpm for 48 h to obtain the fermentation broths.

The above fermentation media contain carbon sources (see Table 4 for combinations and contents of the carbon sources), 13.5 g/L potassium dihydrogen phosphate, 4.0 g/L diammonium hydrogen phosphate, 1.7 g/L citric acid, 1.4 g/L magnesium sulfate heptahydrate and 10 ml/L trace metal elements, the trace metal elements including 10 g/L ferrous sulfate, 2.25 g/L zinc sulfate heptahydrate, 1.0 g/L anhydrous copper sulfate and 2.0 g/L calcium chloride dihydrate, and the pH of the fermentation media being 6.8.

Table 4 shows the results of the yields of lacto-N-neotetraose of the strain A5 in the fermentation media of different combinations of carbon sources. From the table, when the combination of carbon sources in the medium was 10 g/L galactose+10 g/L glycerin, the yield of lacto-N-neotetraose was increased by 23.2% compared to that fermented by using 20 g/L glycerin as the carbon source at the very start.

TABLE 4
Details of combinations of carbon sources in the media

| | Yield of |
|---|---|
| | lacto-N- |
| | neotetraose |
| Carbon source composition in the media | (mg/L) |
| 20 g/L Glycerin | 837 |
| 20 g/L Galactose | 642 |
| 20 g/L Glucose | 402 |
| 10 g/L Glycerin | 701 |
| 10 g/L Galactose | 794 |
| 10 g/L Glucose | 540 |
| 10 g/L Glycerin + 10 g/L Glucose | 217 |
| 10 g/L Galactose + 10 g/L Glycerin | 1031 |
| 10 g/L Glucose + 10 g/L Galactose | 161 |

Although the disclosure has been disclosed as above in preferred embodiments, it is not intended to limit the disclosure. Anyone familiar with this technology can make various changes and modifications without departing from the spirit and scope of the disclosure. Therefore, the protection scope of the disclosure should be defined by the claims.

1. Genetically engineered bacteria producing lacto-N-neotetraose, wherein in the genetically engineered bacteria, β-galactosidase gene lacZ is knocked out, and β-1,3-acetylglucosamine transferase gene lgtA, β-1,4-galactosyltransferase gene lgtB, phosphoglucomutase gene pgm, UDP-glucose-4-epimerase gene galE, galactose-1-phosphate uridyltransferase gene galT, galactokinase gene galK and β-galactoside permease gene lacY are overexpressed.

2. The genetically engineered bacteria according to claim 1, wherein the β-1,3-acetylglucosamine transferase gene lgtA and β-1,4-galactosyltransferase gene lgtB are both from Neisseria meningitidis; the nucleotide sequence of β-1,3-acetylglucosamine transferase gene lgtA is set forth in SEQ ID NO: 1; and the nucleotide sequence of β-1,4-galactosyltransferase gene lgtB is set forth in SEQ ID NO: 2.

3. The genetically engineered bacteria according to claim 1, wherein the phosphoglucomutase gene pgm, UDP-glucose-4-epimerase gene galE, galactose-1-phosphate uridyltransferase gene galT, galactokinase gene galK, β-galactoside permease gene lacY and β-galactosidase gene lacZ are all from Escherichia coli K-12; the nucleotide sequence of phosphoglucomutase gene pgm is set forth in SEQ ID NO: 24, the nucleotide sequence of UDP-glucose-4-epimerase gene galE is set forth in SEQ ID NO: 25, the nucleotide sequence of galactose-1-phosphate uridyltransferase gene galT is set forth in SEQ ID NO: 26, the nucleotide sequence of galactokinase gene galK is set forth in SEQ ID NO: 27, the nucleotide sequence of β-galactoside permease gene lacY is set forth in SEQ ID NO: 28, and the nucleotide sequence of β-galactosidase gene lacZ is set forth in SEQ ID NO: 23.

4. The genetically engineered bacteria according to claim 1, wherein the genetically engineered bacteria express the gene lacY with pETDuet-1 plasmid, express the genes pgm, galE, galT and galK sequentially with pRSFDuet-1 plasmid, and express the genes lgtA and lgtB sequentially with pCDFDuet-1 plasmid.

5. A method of use of the genetically engineered bacteria of claim 1 for producing lacto-N-neotetraose, comprising the following steps:
(1) culturing the genetically engineered bacteria in a seed medium to obtain a seed solution;
(2) inoculating the seed solution into a fermentation system and culturing the seed solution until the OD₆₀₀ is 0.6-0.8; and
(3) adding IPTG with a final concentration of 0.4 mM and lactose with a final concentration of 8 g/L-10 g/L for performing induction to obtain a fermentation broth containing lacto-N-neotetraose.

6. The method according to claim 5, wherein carbon source in the fermentation system is one or more of glucose, galactose and glycerin.

7. The method according to claim 6, wherein the carbon source is 20 g/L glycerin, or a mixture of 10 g/L glycerin and 10 g/L galactose; and the fermentation system further contains 13.5 g/L potassium dihydrogen phosphate, 4.0 g/L diammonium hydrogen phosphate, 1.7 g/L citric acid, 1.4 g/L magnesium sulfate heptahydrate and 10 ml/L trace metal elements, the trace metal elements comprising 10 g/L ferrous sulfate, 2.25 g/L zinc sulfate heptahydrate, 1.0 g/L anhydrous copper sulfate and 2.0 g/L calcium chloride dihydrate.

8. The method according to claim 5, wherein the seed medium in the method is an LB liquid medium, and the seeds are cultured at 35° C.-40° C. and 200 rpm-250 rpm for 10-14 hours in a shake flask.

9. The method according to claim 5, wherein the seed solution is inoculated into the fermentation system and is cultured at 35° C.-40° C. and 200 rpm-250 rpm until the OD₆₀₀ is 0.6-0.8, and culture is induced at 22° C.-30° C. and 200 rpm-250 rpm for 42-48 hours.
